(19) United States
(12) Patent Application Publication
(10) Pub. No.: US 2009/0271915 A1
(43) Pub. Date: Nov. 5, 2009

(54) Dynamic Scenery Swim Mask

(76) Inventor: Nicholas Ross, St. Petersburg, FL (US)

Correspondence Address: Maxey Law Offices, PLLC, 13630 58TH ST. NORTH, SUITE 101, CLEARWATER, FL, 33760, US

(21) Appl. No.: 12/112,091

(22) Filed: Apr. 30, 2008

(51) Int. Cl. A61F 9/02

(52) U.S. Cl. 2/428

(57) ABSTRACT

A dynamic scenery swim mask with a liquid filled lens having a plurality of decorative elements. The lens includes a rearward lens element and a forward lens element defining a sealed fluid-receiving compartment therebetween. The rearward lens element having a first viewing pane with a peripheral edge and a continuous sidewall extending forwardly from said peripheral edge. The forward lens element having a second viewing pane and being sealingly attached to the sidewall at a forwardly spaced distance from said first viewing pane. The fluid receiving compartment is filled with a flowable fluid and one or more decorative elements.

### FIELD OF THE INVENTION

The present invention relates generally to a swim mask, and more particularly, relating to a dynamic scenery swim mask having a fluid filled lens containing a plurality of free moving decorative elements within the field of vision of a user.

### BACKGROUND OF THE INVENTION

Swim masks of numerous constructions to serve an equal number of purposes are known. However, heretofore there exists no swim mask which provides a dynamic scenery within the field of vision of the user permitting an enhanced and a virtual visual experience that simulates an underwater environment, stimulates imagination, promotes pretend play, and is entertaining.

Children learn by imagining and doing. Research has shown that pretend play provides children with a microcosm for life that encourages them to take the skills they have learned and apply them to meaningful life activities. It is believed that this process of application helps children not only develop a skill, but learn how to use it in life. Imagination helps school-age children solve problems by helping them think through different outcomes to various situations and role playing ways to cope with difficult or new circumstances.

U.S. Pat. No. 5,103,145 to Croll discloses eyeglasses having decorative lenses including a parallel double lens system rigidly mounted in a frame creating an enclosed space between the lenses which is filled with a flowable liquid. Ports through the eyeglass frame into the enclosed space provide for filling of the lens with the flowable liquid and the introduction of decorative elements, including floatable and non-floatable decorative materials. While the lens system of the Croll eyeglasses meets its particular objectives and requirements, the construction of the lens system inherently does not permit under water use where the lens system would be subjected to pressures above atmosphere, which would likely result in the flowable liquid leaking from the enclosed space.

Accordingly, there is a need for swim mask with a new lens construction with a sealed enclosed space which is filled with a flowable liquid and decorative elements that is not subject to leaking at pressures above atmosphere, thereby providing a swim mask with a dynamic scenery which provides a user with an enhanced and a virtual visual experience that simulates an underwater environment, stimulates imagination, promotes pretend play, and is entertaining.

### SUMMARY OF THE INVENTION

The preferred embodiments of the present invention address these needs by providing a swim mask having dynamic scenery with a new fluid filled lens construction.

It is an objective of the present invention to provide a swim mask with dynamic scenery for visual enhancement of a user.

It is another objective of the present invention to provide a new liquid filled lens with an improved construction capable of with standing pressures higher than atmospheric pressure.

It is another objective of the present invention to provide a swim mask with having a liquid filled lens with a plurality of decorative elements to simulate flora and fauna of an underwater environment.

It is another objective of the present invention to provide a swim mask which promotes imagination and pretend play by the user.

To achieve these and other advantages, in general, in one aspect, a dynamic scenery swim mask is provided. The mask has a lens including a rearward lens element and a forward lens element defining a sealed fluid-receiving compartment therebetween. The rearward lens element has a first viewing pane with a peripheral edge and a continuous sidewall extending forwardly from the peripheral edge. The forward lens element has a second viewing pane and is sealingly attached to the sidewall at a forwardly spaced distance from the first viewing pane. A skirt having a forward annular receiving portion with a rearward flange and a forward flange. The lens is positioned within the forward annular receiving portion between the forward flange and said rearward flange. A frame having the skirt being inserted into the frame. A flowable fluid within the fluid-receiving compartment. One or more decorative elements within said fluid-receiving compartment.

In general, in another aspect, the one or more decorative elements are freely moveable within the fluid-receiving compartment.

In general, in another aspect, the one or more decorative elements includes at least one neutrally buoyant decorative element and at least one negatively buoyant decorative element.

In general, in another aspect, the flowable fluid contains a first fluid of a first density and a second fluid of a second density greater than the first density.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is a perspective view of the dynamic scenery swim mask constructed in accordance with the principles of the present invention;

FIG. 2 is a side cross sectional view of the dynamic scenery swim mask showing the assembly details of the mask and the construction of the fluid filled lens;

FIG. 3 is a front elevation view of the mask illustrating one aspect of the decorative elements and fluid filled lens;

FIG. 4 is a front elevation view of the mask illustrating a second aspect of the decorative elements and fluid filled lens; and

FIG. 5 is a front elevation view of the mask illustrating a third aspect of the decorative elements and fluid filled lens.

### DETAILED DESCRIPTION OF THE INVENTION

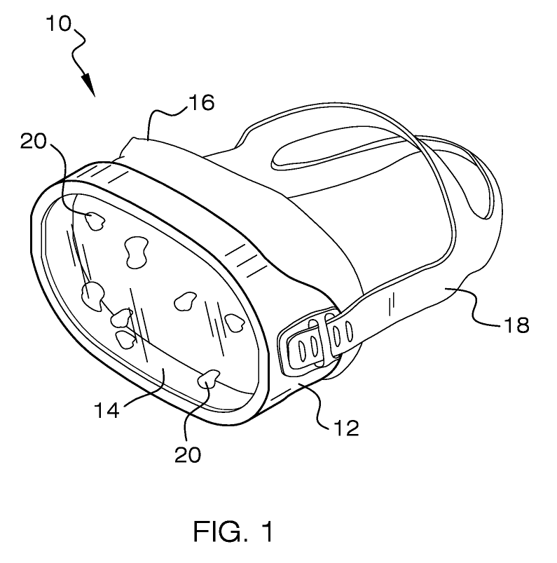

Reference will now be made in detail to the preferred embodiments of the present invention, examples of which are illustrated in the accompanying drawings. In FIG. 1 a new swim mask 10 of the present invention for providing dynamic scenery to a user while swimming is illustrated and will be described. The construction of the swim mask 10 is much like the construction of a conventional swim mask and includes a frame 12, a lens 14, a skirt 16 and an adjustable head securing strap 18. However, unlike conventional swim masks, the lens 14, of a new construction, is fluid-filled and contains a plurality of freely movable decorative elements 20 to enhance a users visual experience while wearing the mask, for example while swimming underwater. The decorative elements 20 may be constructed to simulate various underwater flora and fauna.

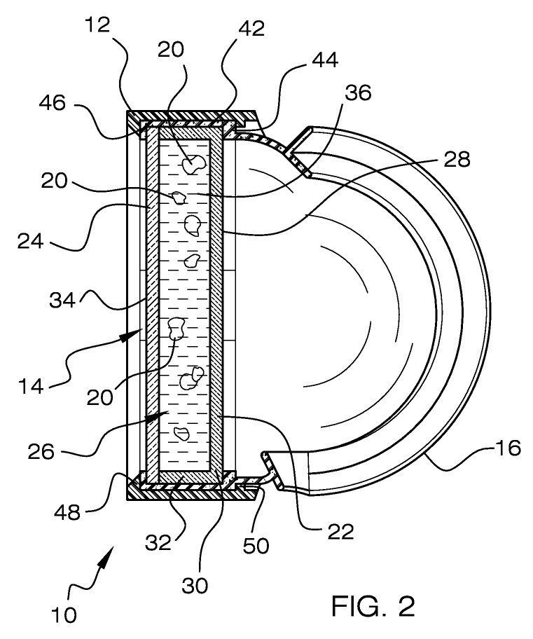

With further reference to FIG. 2, the lens 14 includes a rearward lens element 22 and a forward lens element 24 defining a fluid-receiving compartment 26 therebetween. The rearward lens element 22 includes a first viewing pane 28 with a peripheral edge 30 from which a continuous sidewall 32 forwardly extends. The forward lens element 24 has a second viewing pane 34 and is sealingly attached to the sidewall 32 with the second viewing pane at forwardly spaced distance from the first viewing pane 28. The lens 14 is free from access ports into the fluid receiving compartment 26. Thus, before the forward lens element 24 is sealingly attached to the rearward lens element 22, one or more decorative elements 20 and a flowable fluid 36 is introduced into the fluid-receiving compartment 26. Then, the forward lens element 24 is hermetically sealed to the rearward lens element 22 preventing fluid and air from entering or exiting the fluid-receiving compartment 26. The fluid 36 may be a translucent fluid which may have an desired degree of coloration. The fluid 36 may be colored by the addition of dyes. The fluid 36 may contain two or more fluids of different densities to achieve a layered or two-toned effect.

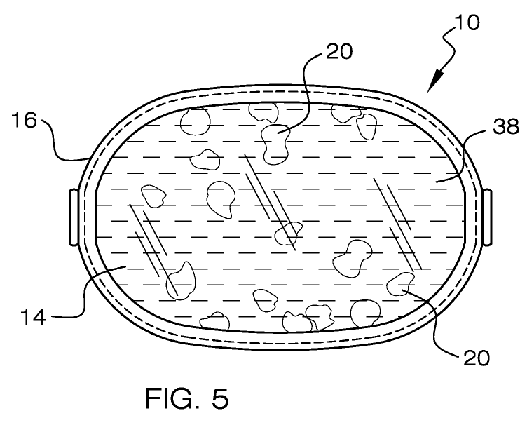

The decorative elements 20 may be neutrally buoyant, negatively buoyant (non-floatable), positively buoyant (floatable) or any combinations thereof, as shown in FIG. 5. The elements 20 may have various decorative or simulate shapes, such as, but not limited to, fish, starfish, sea shells, coral, sea fans, crabs, sea urchins and other underwater objects and the like. The decorative elements 20 are independent and freely movable within the fluid 36, meaning they are not secured or otherwise tethered or attached to any portion of the swim mask 10, such that when a user moves his or her head, the decorative elements may be caused to move. Movement of the decorative elements 20 may be viewed from the perspective of a user as being relative to the surroundings or environment of the user.

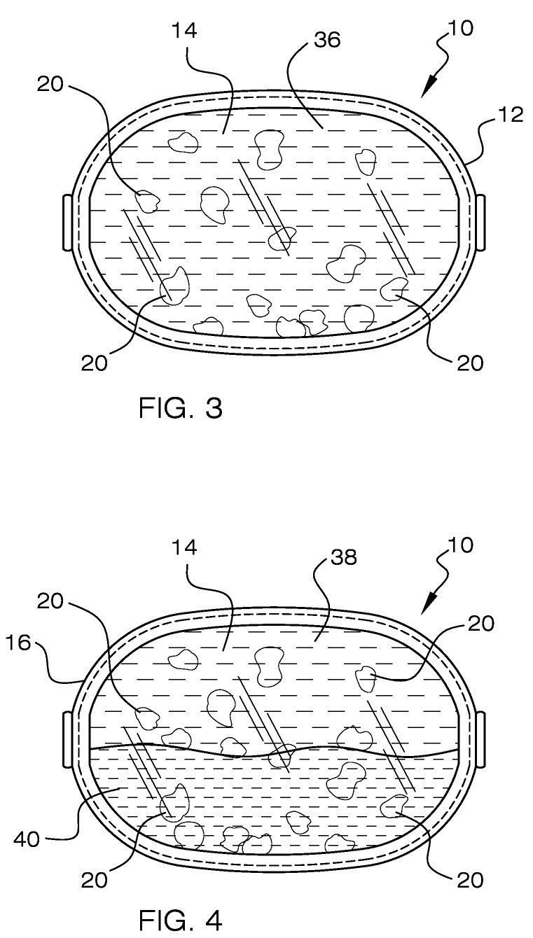

With reference to FIG. 3, in one aspect, each of the decorative elements 20 are selected to be neutrally buoyant in the flowable fluid 36. In FIG. 4, in a second aspect, the flowable fluid 36 includes a first fluid 38 of a first density and a second fluid 40 of a second density greater than that of the first fluid, thereby creating a two-toned or layered effect. The first fluid 38 may be oil or a fluid containing oil. The second fluid 40 may be water or a fluid containing water. In this aspect, and as illustrated, the decorative elements 20 can be selected to be neutrally buoyant, negatively buoyant or positively buoyant with respect to either the first or second fluids 38, and 40. Specifically, one or more decorative elements 20 can be selected to be positively buoyant with respect to the second fluid 40 and negatively buoyant with respect to the first fluid 38, such that the decorative element is caused to float along the interface of the two fluids. While the fluid-receiving compartment 26 is shown as being substantially filled with the flowable fluid 36, alternatively, the fluid-receiving compartment may be partially filled with the flowable fluid 36.

Turning back to FIG. 2, the frame 12 may be formed of a rigid or semi-rigid material. The skirt 16 is formed of an elastomeric material, such as silicon rubber. The lens 14 may be formed of glass or a suitable plastic. The lens 14 may be attached or otherwise secured to the skirt 16 by over-molding to form a portion covering the face including the eyes and/or nose and having a waterproof seal. It is contemplated that the skirt 16 and the lens 14 can be attached together by other techniques, such as friction, or adhesively. The skirt 16 has a forward annular receiving portion 42 with a rearward flange 44 and a forward flange 46 into which the lens 14 is fitted with the forward lens element 24 positioned forwardly of the rearward lens element 22. The frame 12 has two separate annular flanges 48 and 50 into which the skirt 16 and lens 14 combination is fitted. The construction of the lens 14 and the assembly of the mask 10 ensures that the fluid 36 is prevented from getting into the users eyes should the seal between the forward lens element 24 and rearward lens element 22 fails.

The head securing strap 18 is a conventional head strap used in the art for securing swim masks or swim goggles to the face of a user. The head securing strap may be adjustable secured to the frame 12 by any conventional or known techniques in the field.

A number of embodiments of the present invention have been described. Nevertheless, it will be understood that various modifications may be made without departing from the spirit and scope of the invention. Accordingly, other embodiments are within the scope of the following claims.

What is claimed is:

1. A dynamic scenery swim mask comprising:
a lens including a rearward lens element and a forward lens element defining a sealed fluid-receiving compartment therebetween;
said rearward lens element having a first viewing pane with a peripheral edge and a continuous sidewall extending forwardly from said peripheral edge;
said forward lens element having a second viewing pane and being sealingly attached to said sidewall at a forwardly spaced distance from said first viewing pane;
a skirt having a forward annular receiving portion with a rearward flange and a forward flange;
said lens being positioned within said forward annular receiving portion between said forward flange and said rearward flange;
a frame, said skirt being inserted into said frame;
a flowable fluid within said fluid-receiving compartment; and
one or more decorative elements within said fluid-receiving compartment.

2. The dynamic scenery swim mask of claim 1, wherein said one or more decorative elements are freely moveable within said fluid-receiving compartment.

3. The dynamic scenery swim mask of claim 1, wherein said one or more decorative elements are neutrally buoyant.

4. The dynamic scenery swim mask of claim 1, wherein said one or more decorative elements includes at least one neutrally buoyant decorative element and at least one negatively buoyant decorative element.

5. The dynamic scenery swim mask of claim 1, wherein said flowable fluid contains a first fluid of a first density and a second fluid of a second density greater than the first density.

6. The dynamic scenery swim mask of claim 5, wherein said first fluid comprises oil.

7. The dynamic scenery swim mask of claim 5, wherein said second fluid is comprises water.
